(19) United States
(12) Plant Patent Application Publication
(10) Pub. No.: US 2013/0247265 P1
(43) Pub. Date: Sep. 19, 2013

(54) Agapanthus plant named 'HinesAg1'

(50) ;

(71) Applicant: David Hernandez, Orange, CA (US)

(72) Inventor: David Hernandez, Orange, CA (US)

(73) Assignee: Hines Growers LLC, Laguna Hills, CA (US)

(21) Appl. No.: 13/385,985

(22) Filed: Mar. 19, 2012

(51) Int. Cl. A01H 5/00

(52) U.S. Cl. PLT/398

(57) ABSTRACT

An Agapanthus plant particularly distinguished by large, bluish-purple inflorescences, and yellow and green variegated foliage, is disclosed.

### GENUS AND SPECIES

Agapanthus africanus

### VARIETY DENOMINATION

‘HinesAgl’

### BACKGROUND OF THE NEW PLANT

The present invention comprises a new and distinct variety of Agapanthus, botanically known as Agapanthus africanus, and hereinafter referred to by the variety name ‘HinesAgl’. The new variety originated as a sport from tissue culture of Agapanthus africanus ‘Hinag’, commercially known as ‘Summer Gold’ (U.S. Plant Pat. No. 10,866) in June 1999 in Irvine, Calif.

The new plant was first asexually propagated in Irvine, Calif. in 1999 and has been asexually reproduced by divisions for approximately 12.5 years in Irvine, Calif. The present invention has been found to retain its distinctive characteristics through successive asexual propagations by divisions.

‘HinesAgl’ has not been made publicly available or sold more than one year prior to the filing of this application.

### SUMMARY

The following are the most outstanding and distinguishing characteristics of the new variety when grown under normal horticultural practices in Irvine, Calif.

1. Large, bluish-purple inflorescences; and

2. Yellow and green variegated foliage.

### DESCRIPTION OF THE PHOTOGRAPHS

This new Agapanthus plant is illustrated by the accompanying photographs which show the overall plant habit, foliage and inflorescence of the plant. The colors are as true as can be reasonably obtained by conventional photographic procedures. The photographs are of a 3-year old plants grown outdoors in Irvine, Calif. in May 2010.

FIG. 1 shows a the whole plant, including the habit, foliage and inflorescence.

FIG. 2 shows a close-up of the foliage.

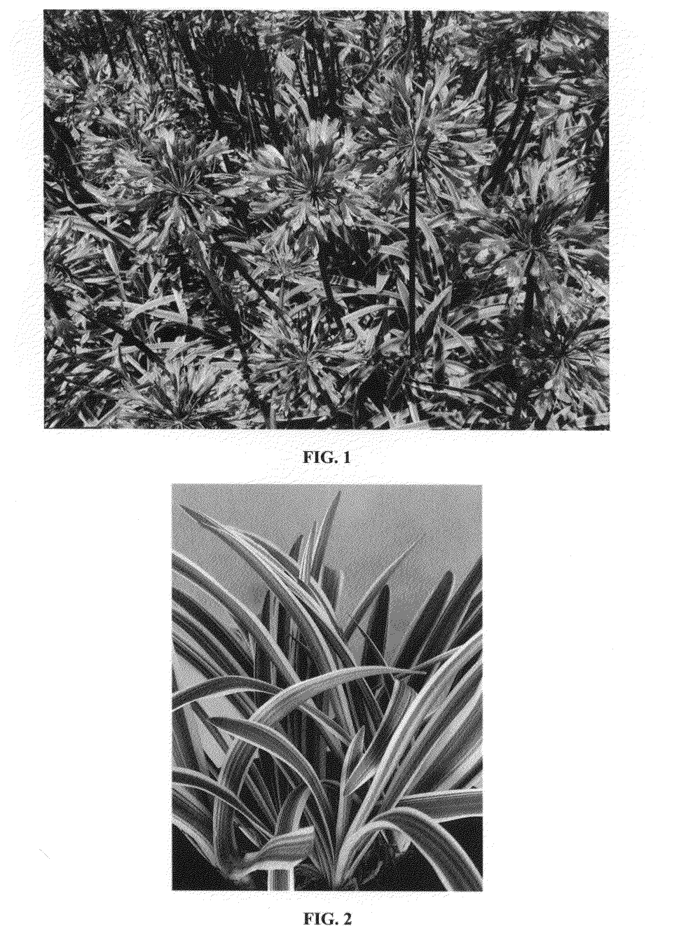

### DESCRIPTION OF THE NEW VARIETY

The following detailed descriptions set forth the distinctive characteristics of ‘HinesAgl’. The data which defines these characteristics were collected from asexual reproductions carried out in Irvine, Calif. The plant history was taken in January 2012 on 1-year old plants grown outdoors in Irvine, Calif. Color references were taken under natural light and are to the R.H.S. Colour Chart of The Royal Horticultural Society of London (R.H.S.), fifth edition (2007). Texture descriptions were taken using a dissecting microscope.
Classification:
   Family.—Amaryllidaceae.
   Botanical name.—Agapanthus africanus.
   Common name.—African lily.
   Denomination.—‘HinesAgl’.
Parentage:
   Parent.—Agapanthus africanus ‘Hinag’, commercially known as ‘Summer Gold’ (U.S. Plant Pat. No. 10,866).
Plant:
   Propagation type.—Divisions.
   Plant height.—31.0 cm.
Foliage:
   Quantity of blades per plant.—11.
   Arrangement.—Basal.
   Length.—45.7 cm.
   Width.—3.2 cm to 3.8 cm.
   Shape of blade.—Linear.
   Margin.—Entire.
Inflorescence:
   Height.—88.9 cm.
   Width.—14.0 cm.
   Petals.—Arrangement and quantity per flower: Seven arranged in a single whorl with the petals fused into a narrow tube Length: 2.5 cm Width: 1.0 cm Base: Fused.
Reproductive organs:
   Stamens.—Quantity per flower: 7.
Comparison with parental and commercial variety: ‘HinesAgl’ differs from the parent ‘Hinag’ (U.S. Plant Pat. No. 10,866) in that ‘HinesAgl’ has a taller plant height (31.0 cm) than ‘Hinag’ (about 18.0 cm). ‘HinesAgl’ differs from the commercial comparison ‘Befran’ (U.S. Plant Pat. No. 21,705) in that ‘HinesAgl’ has a longer leaf blade (45.7 cm) than ‘Benfran’ (30.0 cm).

I claim:

1. A new and distinct variety of Agapanthus plant as shown and described herein.
